(19) United States
(12) Patent Application Publication
(10) Pub. No.: US 2017/0311866 A1
(43) Pub. Date: Nov. 2, 2017

(54) SOFT TISSUE MANAGEMENT METHOD AND SYSTEM

(71) Applicant: RMIT University, Melbourne, Victoria (AU)

(72) Inventors: Franz Konstantin FUSS, Bundoora, Victoria (AU); Aaron BELBASIS, Bundoora, Victoria (AU)

(21) Appl. No.: 15/522,544

(22) PCT Filed: Oct. 30, 2015
(86) PCT No.: PCT/AU2015/000652

§ 371 Date: Apr. 27, 2017

(30) Foreign Application Priority Data
Oct. 31, 2014 (AU) 2014904381

(51) Int. Cl. A61B 5/22; G09B 5/00; A63B 24/00; A61B 5/11; A61B 5/0488; A61B 5/00; G09B 19/00

(52) U.S. Cl. CPC A61B 5/22; G09B 19/003; G09B 5/00; A63B 24/0062; A61B 5/746; A61B 5/0488; A61B 5/4585; A61B 5/4528; A61B 5/1118; A61B 2503/10

(57) ABSTRACT

A method is provided for monitoring and managing muscle activity and soft tissue loading. The method includes providing to a subject a plurality of sensors for measuring muscle activity and soft tissue loading levels; directing the subject to undertake a program of exercise; measuring muscle activity and soft tissue loading during the program of exercise; comparing the measured muscle activity and soft tissue loading levels against calibrated muscle activity and soft tissue loading levels for the subject; and alerting the subject if the comparison of measured muscle activity and soft tissue loading levels against calibrated muscle activity and soft tissue loading levels indicates that a desirable level of muscle activity and/or soft tissue loading is being exceeded.

### TECHNICAL FIELD

The present invention relates to methods and systems for monitoring and managing muscle activity and soft tissue loading. More particularly, the invention relates to methods and systems for monitoring muscle activity and soft tissue loading during exercise with a view to reducing the risk of injury.

### BACKGROUND OF INVENTION

Soft tissue injuries to muscles and ligaments are among the most common sports injuries. Typically such injuries are sustained from repeated action such as long-distance jogging which may be termed as chronic overuse, as opposed to acute injuries, which occur in an instant, such as a sprained ankle or a ruptured cruciate ligament.

Exercise applies stresses to the body to which the body adapts by thickening and strengthening the tissues involved. This results in muscles becoming stronger, firmer and sometimes larger, tendons and ligaments getting stronger and an increase in bone density. However, if exercise is applied in such a way that adaptation to the stresses imparted by exercise cannot occur, then excessive overload can cause microscopic injuries, leading to inflammation. More serious acute injuries can result in the patient to take extended leave from their training program. Accordingly, soft tissue injuries are particularly inconvenient in the case of professional sportspersons, such as for example, AFL (Australia Football League) players.

Many soft tissue injuries could be prevented, particularly where training that takes place in a controlled environment such as the gym where the movement, load and duration of loading applied to the body are readily controlled. Soft tissue injuries occur when the load on the tissue is greater than the “tolerance” or load the tissue can bear.

While even proper movement may result in excessive soft tissue loading, that is in the case of a chronic overuse injury for example, typically excessive tissue loading occurs due to poor movement patterns; poor training load management, i.e. fatigue; and/or poor technique. Accordingly, it would be highly desirable to provide a method and system for monitoring muscle and ligament activity during a training session, to better understand and avoid the risk of soft tissue injury.

### SUMMARY OF INVENTION

According to an aspect of the present invention, there is provided a method for monitoring and managing muscle activity and soft tissue loading, the method including the following steps: (a) providing to a subject a plurality of sensors for measuring muscle activity and soft tissue loading levels; (b) directing the subject to undertake a program of exercise; (c) measuring muscle activity and soft tissue loading during the program of exercise; (d) comparing the measured muscle activity and soft tissue loading levels against calibrated muscle activity and soft tissue loading levels for the subject; and (e) alerting the subject if the comparison of measured muscle activity and soft tissue loading levels against calibrated muscle activity and soft tissue loading levels indicates that a desirable level of muscle activity and/or soft tissue loading is being exceeded.

In an embodiment, the step of providing to a subject a plurality of sensors for measuring muscle activity and soft tissue loading levels includes providing at least two sensors configured to measure muscle activity and at least one sensor configured to measure a joint angle of a joint proximal to a muscle whose activity is to be measured by the at least two sensors. The soft tissue loading levels may be determined as a function of the flexion angle of the proximal joint.

The angle (θ_{ACL}) of an anterior cruciate ligament (ACL) of a lower limb to the tibial plateau may be expressed as a function of the knee flexion angle θ_{KF}, θ_{ACL}=f (θ_{KF}) and anterior cruciate ligament forces (F_{ACL}) may be determined from an angle of the anterior cruciate ligament such that F_{ACL}=Fₓ₋ₙₑₜ/cos θ_{ACL}, wherein F x₋ₙₑₜ is a horizontal net force determined as a sum of horizontal force components of a patellar ligament, hamstrings, and external force, applied by a ground surface to the lower limb.

The angle (θ_{ACL}) of a posterior cruciate ligament (PCL) of a lower limb to the tibial plateau may be expressed as a function of the knee flexion angle θ_{KF}, θ_{PCL}=f (θ_{KF}) and posterior cruciate ligament forces (F_{PCL}) may be determined from an angle of the posterior cruciate ligament such that F_{PCL}=(−1)Fₓ₋ₙₑₜ/cos θ_{PCL}, wherein F x₋ₙₑₜ is a horizontal net force determined as a sum of horizontal force components of a patellar ligament, hamstrings, and external force, applied by a ground surface to the lower limb.

In another embodiment, a simultaneous contraction of agonist and antagonist muscles may be determined from a differential of muscle forces such that CC=F_{Q}−F_{H} wherein, F_{Q}=quadriceps force and F_{H}=hamstrings force as determined from sensed voltage signals.

In a preferred for of the present invention, the step of comparing the measured muscle activity and soft tissue loading levels against calibrated muscle activity and soft tissue loading levels occurs in real-time. Furthermore, the step of alerting the subject if the comparison of measured muscle activity and soft tissue loading levels against calibrated muscle activity and soft tissue loading levels indicates that a desirable level of muscle activity and/or soft tissue loading is being exceeded preferably occurs in real-time.

In a particular embodiment, determination of the calibrated muscle activity and soft tissue ligament loading levels includes directing the subject to perform a series of movements and measuring the muscle activity and soft tissue loading levels of the subject for each movement to build a baseline profile for the subject against which muscle activity and soft tissue loading levels measured during a program of exercise will be compared.

The step of calibrating the muscle activity and soft tissue loading levels for the subject may involve measuring a maximum voluntary contraction of a quadricep and a hamstring respectively corresponding to at least three different knee flexion angles.

The step of alerting the subject if the comparison of measured muscle activity and soft tissue loading levels against calibrated muscle activity and soft tissue loading levels indicates that a desirable level of muscle activity and/or soft tissue loading levels is being exceeded may include providing an auditory, visual or tactile alert to the subject.

In one particular embodiment, the step of providing to a subject a plurality of sensors for measuring muscle activity and soft tissue loading levels involves providing a garment incorporating the sensors to the subject.

According to another aspect of the present invention, there is provided a system for monitoring and managing muscle activity and soft tissue loading, the system including: (a) a plurality of sensors for measuring electric signals indicative of muscle activity and soft tissue loading levels; (b) a processor configured to receive the electric signals and covert them to muscle activity and soft tissue loading values, the processor further configured to compare the muscle activity and soft tissue loading values against calibrated muscle activity and soft tissue loading levels for a subject; and (c) an alert module to alert the subject if the comparison of measured muscle activity and/or soft tissue loading levels against calibrated muscle activity and soft tissue loading levels indicates that a desirable level of muscle activity and/or soft tissue loading is being exceeded.

The plurality of sensors for measuring electric signals indicative of muscle activity and soft tissue loading levels may include at least three sensors.

The at least three sensors are may be positioned on the subject at the following locations: (a) a first position which will contact an anterior, medial or posterior skin surface of a body segment of the subject; (b) a second position which will contact a remaining contact an anterior, medial or posterior skin surface of the body segment; and (c) a third position which will contact an anterior, posterior, medial or lateral skin surface of a joint proximal to the body segment. The sensors in the first and second positions may be configured to measure muscle activity of the body segment. Furthermore, the sensor in the third position may be configured to measure the angle of the joint proximal to the body segment.

The plurality of sensors may include a combination of pressure sensors and electrogoniometric sensors.

In an embodiment, the plurality of sensors for measuring electric signals indicative of muscle activity and soft tissue loading levels are incorporated into a garment to be donned by the subject. The garment may be a compression garment.

According to yet another aspect of the present invention, there is provided a training aid for monitoring and managing muscle activity and soft tissue loading, the training aid including: (a) a garment incorporating a plurality of sensors for measuring electric signals indicative of muscle activity and soft tissue loading levels; (b) processor configured to receive the electric signals and covert them to muscle activity and soft tissue loading values, the processor further configured to compare the muscle activity and soft tissue loading values against calibrated muscle activity and soft tissue loading levels for a subject; and (c) an alert module to alert the subject if the comparison of measured muscle activity and/or soft tissue loading levels against calibrated muscle activity and soft tissue loading levels indicates that a desirable level of muscle activity and/or soft tissue loading is being exceeded.

The processor and alert module may be provided in a portable telecommunications device.

The garment incorporating a plurality of sensors may be a compression garment.

### BRIEF DESCRIPTION OF DRAWINGS

The invention will now be described in further detail by reference to the accompanying drawings. It is to be understood that the particularity of the drawings does not supersede the generality of the preceding description of the invention.

FIG. 1 shows a flow chart showing generally the steps of a method embodying the present invention.

FIG. 2 is a schematic diagram of a system for performing the method described with reference to FIG. 1.

FIG. 3 is a schematic diagram showing various functional elements of a computer-enabled system for performing the method of the present invention in block form.

FIG. 4 is a photograph of a functional prototype of a training aid according to an embodiment of the present invention applied to a subject.

FIG. 5 is a photograph showing exemplary ancillary components that may be associated with the training aid shown in FIG. 4 during use.

FIGS. 6A to 6C show exemplary voltage signals detected by the sensors secured to a body segment as described with reference to FIG. 4.

FIG. 7 shows an enlarged sample of the data of FIGS. 6A to 6C.

### DETAILED DESCRIPTION

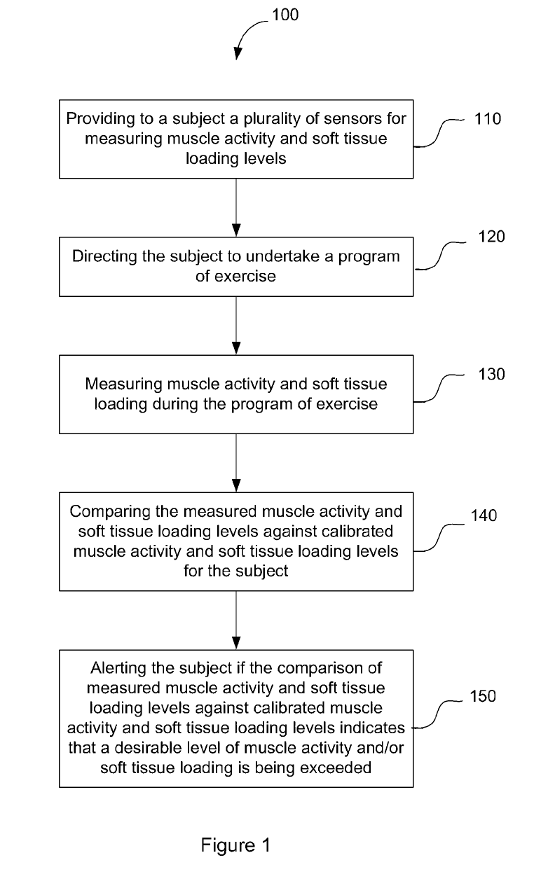

Referring firstly to FIG. 1, there are generally shown the steps of a method for monitoring and managing muscle activity and soft tissue loading according to the invention. The method is intended to monitor muscle activity and soft tissue loading during training and exercise with a view to providing feedback to a subject to lead to an increased level of understating of when soft tissue injuries are likely to occur, and to assist both professional and amateur athletes to avoid such injuries by alerting them when excessive loads or activity levels are measured during training and exercise. The feedback loop provided by the method trains the subject to associate particular movements with excessive muscle activity and/or soft tissue loads, so that the subject can modify and/or avoid those particular movements to reduce the risk of injury.

At step 110, a subject, typically an athlete, whether professional or amateur, is provided with a plurality of sensors to be positioned on one or more body segments as will be later described in more detail. It is to be understood that a body segment may be any part of the body comprising muscle tissue, particularly the limbs and torso. Once the sensors positioned on a body segment, the subject is directed to undertake his or her training program, which may involve a series of exercises, a 5 km jog, or the like, at step 120. While the subject performs the relevant activity, the sensors are activated to measure muscle activity and soft tissue loading at step 130.

At step 140, the muscle activity and soft tissue loading levels measured during the exercise are compared against previously calibrated muscle activity and soft tissue loading levels. The calibrated muscle activity and soft tissue loading levels are unique to the particular subject and effectively embody a baseline profile, deemed to be a safe or desirable activity and loading level, against which future activity and loading levels will be evaluated.

If the comparison of the measured muscle activity and soft tissue loading levels against the calibrated levels indicates that the measured values are exceeding the calibrated levels, either in terms of the muscle activity or the soft tissue loading, then an alert will generate to notify the subject at step 150.

One particular advantage of the method for monitoring muscle activity and soft tissue loading is that the comparison of the measured muscle activity and soft tissue loading levels against calibrated muscle activity and soft tissue loading levels can occur in real-time. Likewise, if the comparison indicates that one of or both of the measured muscle activity and soft tissue loading levels exceed the calibrated levels, then an alert can be generated in real-time to notify the subject. This enables the subject to receive virtually instantaneous feedback as they perform the movement or exercise causing the muscle activity or soft tissue loading levels to exceed desirable levels. Accordingly, the subject will rapidly learn that a particular exercises or movement which generates an alert during training should be modified, for example, by reducing intensity or repetition, or by an improvement in form, or alternatively avoided altogether to reduce the risk of injury.

The alert provided at step 150 may be an auditory, visual or tactile alert such as a vibration.

To provide useful data, the plurality of sensors should include at least two sensors of a type to measure muscle activity levels, such as pressure or force sensors configured to measure electrical signals based on the increase in muscle volume during contraction. That is, the sensors increase their resistance or capacitance with increasing compression or tension in the muscle proximal to their position. The sensors may be pressure/force sensors that are integrated into a strap or textile (e.g. conductive materials or structures, such as conductive fabrics) which change their resistance or capacitance with increasing compression, or stretch sensors similarly integrated into a strap or textile (e.g. conductive materials or structures such as conductive fabrics incorporating strain gauges) which change their resistance or capacitance with increasing tension.

Whilst electromyography or EMG sensors may be suitable in some applications of the method, such as in a highly controlled laboratory or clinical environment, they are not the preferred sensor type for everyday training applications in the field due to their inherent requirement for relatively accurate placement on the midline of the muscle whose activity level is to be measured, in order to obtain valid and repeatable results.

One example of a suitable sensor is described in Australian Patent Application No. 2013902584 the contents of which are incorporated herein by reference. The sensor may comprise a sensor array provided in a material having resistant, capacitive or piezoelectric properties which react to various surface pressures.

At least one additional sensor of an alternative type is employed to measure the angle of a joint which is proximal to the muscle whose activity level is to be measured by sensors described above. One example of a suitable sensor type for measuring joint angles is an electrogoniometry or EGM sensor.

To provide a baseline profile for a particular subject, the first time that the sensors are applied to a particular subject, an additional calibration step is required to provide the calibrated muscle activity and soft tissue loading levels to which the activity and loading levels measured during training and exercise will be compared. Determining the calibrated muscle activity and soft tissue ligament loading levels involves directing the subject to perform a series of movements and measuring the muscle activity and soft tissue loading levels of the subject for each of those movements.

For example, the method for monitoring and managing muscle activity and soft tissue loading will now be described in more detail by reference to an example, wherein the sensors are positioned on the thigh, i.e. the quadriceps and hamstrings, and the knee joint respectively. For example, measured voltage signals from sensors:
V_{Q}=quadriceps (vastus medialis, lateralis, intermedius; rectus femoris)
V_{H}=hamstrings (biceps femoris, semimembranosus, semitendinosus)
V_{KFA}=knee flexion angle

It is to be understood that the method and system of the present invention are equally applicable to other body segments including the upper arm, i.e. the biceps and triceps together with the elbow joint, or indeed the lower limbs.

In this case, in order to calibrate the system, a maximum voluntary contraction or activity of the quadriceps and hamstrings is measured, at five different knee flexion angles. This provides the calibrated muscle activity levels for a variety of knee flexion angles.

In the same example, in order to calibrate cruciate ligament loading, a maximal voluntary contraction of the quadriceps is measured at maximal knee extension, when standing on the contralateral leg, and a maximal voluntary contraction of the hamstrings when sitting on a stool. These measurements represent the shank unloaded and aligned perpendicularly to ground.

To obtain a representative calibration with the shank loaded and aligned perpendicularly to ground, the back is supported by a wall, and a maximum voluntary contraction of the quadriceps in the loaded leg is measured at slight knee flexion, pushing the foot forward when loading the heel. A maximum voluntary contraction of quadriceps of the loaded leg is measured again at slight knee flexion, pushing the foot forward when loading the toes.

The measured electrical or voltage signals are processed to convert them to estimated force and angle data post calibration:
F_{Q}=quadriceps force
F_{H}=hamstrings force
θ_{KF}=knee flexion angle (extension=0°, active flexion=approximately) 140°)

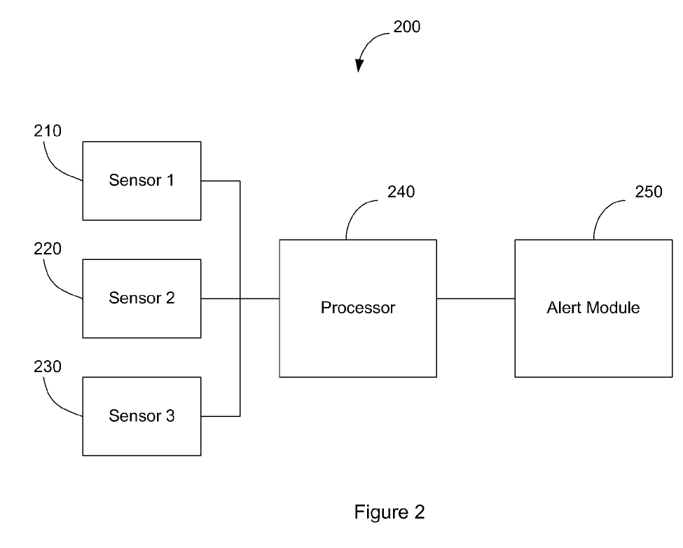

Referring now to FIG. 2, there is shown a system for monitoring and managing muscle activity and soft tissue loading 200. In an embodiment, the system 200 includes at least three sensors 210, 220, and 230 for measuring electric signals indicative of muscle activity and soft tissue loading levels. The three sensors indicated in the system 200 represent the minimum number of sensors attached to a body segment to provide reliable and reproducible results. It should be understood however, that additional sensors may be attached to the same body segment, or to other body segments as required. Increasing the number of sensors employed will provide a greater number of measurements and thereby increase redundancy in the system.

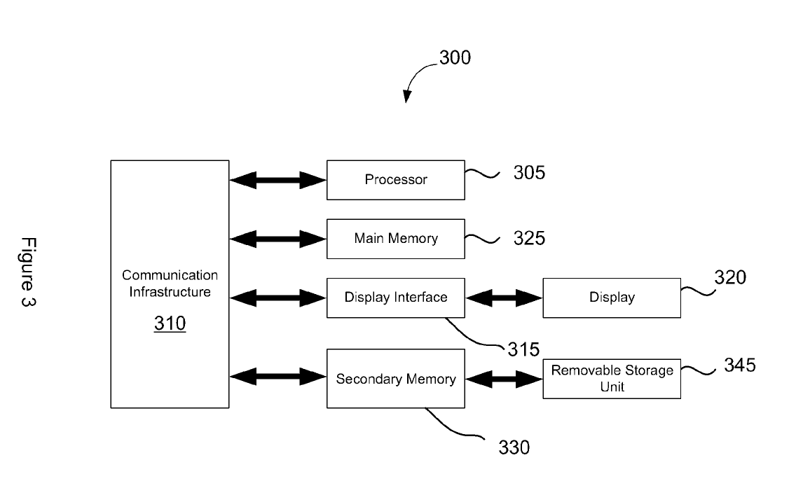

The muscle activity levels and soft tissue loading levels are transmitted to a processor 240 by suitable communication means. The communication means may be tethered or employ wireless protocols and transmitting means between the sensor and the processor. Whilst it is to be understood that the system may be implemented in various ways, the processor 240 or processors may be provided in a standard computing system. Referring now to FIG. 3, the computing system 300 may comprise a portable device such as a laptop or smart phone including one or more processors, a display interface 315 that forwards graphics, texts and other data from a communication infrastructure 310 for supply to the display unit 320. The computing system 300 may also include a main memory 325, preferably random access memory, and may also include a secondary memory 330.

The secondary memory 330 may include a removable storage unit 345 having a computer usable storage medium having stored therein computer software in a form of a series of instructions to cause the processor 305 to carry out the desired functionality described with reference to the method of the invention. In alternative embodiments, the secondary memory 330 may include other similar means for allowing computer programs or instructions to be loaded into the computer system 300.

Referring back to FIG. 2, an alert module 250 provides feedback to the subject where the measured muscle active and/or soft tissue loading levels exceed the calibrated levels. For example, where the computing system is provided by way of a portable computing device such as a laptop, the alert module may produce an auditory or visual alert, wherein the visual alert would be provided on the laptop display. Where the computing system is provided in the form of a more compact portable device such as a smart phone or smart watch, which can be worn by the subject, alternately, the alert could be tactile and/or visual or auditory. That is the smart device worn against the subjects skin can emit a vibration to alert the subject that a desirable or safe level of muscle activity and/or soft tissue loading is being exceeded.

The at least three sensors are positioned on the subject on a body segment at the following locations. At least one sensor is located at the anterior, medial, lateral or posterior skin surface of the body segment of interest. At least one sensor is located at one of the three remaining skin surfaces of the same body segment. And a least one sensor is positioned at the anterior, posterior, medial or lateral skin surface of a joint proximal to the body segment. The sensors positioned on the skin surface of the body segment on the anterior, medial, lateral or posterior surface are configured to measure muscle activity muscle activity, e.g. of antagonistic muscles of the body segment. The sensor positioned proximal to the joint is configured to measure the angle of the joint proximal to the body segment.

For example, continuing with the example of measuring the muscle activity of the thigh and the flexion angle of the proximal knee, at least one sensor may be positioned at the anterior skin surface of thigh; another sensor at the posterior skin surface of thigh; and at least one sensor may be positioned at the anterior, posterior, medial, or lateral skin surface of knee. The sensors positioned at the thigh serve to measure and continuously record muscle activity; while the sensor(s) at the knee serve to measure and record the knee flexion angle.

The processor 240 processes the electrical or voltage signals measured by the sensors 210, 220, 230 in accordance with a series of instructions embodied in software. Now follows a worked example of determining the risk of injury in relation to carious soft tissue structure associated with the thigh/knee body segment example.

In order to determine the risk of cruciate ligament injury, the angle (θ_{ACL}) of anterior cruciate ligament (ACL) to the tibial plateau (positive), is expressed as a function of the knee flexion angle θ_{KF}, θ_{ACL}=f (θ_{KF}), preferably defined by a polynomial fit function. That is:

θ_{ACL}=60.08490163−0.1105096342*θ_{KF}−0.002207774578*pow(θ_{KF},2)+1.189632152E−005*pow(θ_{KF},3)

The angle (θ_{PCL}) of the posterior cruciate ligament (PCL) to the tibial plateau (positive), is expressed as a function of the knee flexion angle θ_{KF}, θ_{PCL}=f (θ_{KF}), preferably defined by a polynomial fit function. That is:

θ_{PCL}=52.07004722−0.1323032773*θ_{KF}+0.004194712106*pow(θ_{KF},2)−1.675160363E−005*pow(θ_{KF},3)

The angle (θ_{PL}) of the patellar ligament with a perpendicular to tibial plateau (positive in extension, negative in flexion), is expressed as a function of the knee flexion angle θ_{KF}, θ_{PL}=f (θ_{KF}), preferably defined by a polynomial fit function. That is:

θ_{PL}=24.11218877−0.09491067242*θ_{KF}−0.004083736642*pow(θ_{KF},2)+2.161222257E−005*pow(θ_{KF},3)

The average angle (θ_{H}) of the hamstrings with a perpendicular to tibial plateau (negative), is expressed as a function of the knee flexion angle θ_{KF}, θ_{H}=f (θ_{KF}), preferably defined by a polynomial fit function. That is:

θ_{H}=−7.619022309−0.4260600571*θ_{KF}−0.00674086388*pow(θ_{KF},2)+2.448438208E−005*pow(θ_{KF},3)

The mechanical advantage MA_{P} of patella is expressed as a function of the knee flexion angle θ_{KF}, MA_{P}=f (θ_{KF}), preferably defined by a polynomial fit function. That is:

MA_{P}=1.399941871−0.005709688462*θ_{KF}+1.04781429E−005*pow(θ_{KF},2)−3.819389092E−006*pow(θ_{KF},X,3)+5.308234954E−008*pow(θ_{KF},4)−1.797478623E−010*pow(θ_{KF},5)

The moment arm (L_{PL}) of patellar ligament (positive), is the shortest distance between the instant centre of the knee and the patellar ligament. The moment arm of patellar ligament is expressed as a function of the knee flexion angle θ_{KF}, L_{PL}=f (θ_{KF}), preferably defined by a polynomial fit function. That is:

L_{PL}=[5.0003127−0.01223030863*θ_{KF}−8.70457433E−005*pow(θ_{KF},2)+7.487734353E−007*pow(θ_{KF},3)]/100

The average moment arm L_{H} of hamstring tendons (negative), is the shortest average distance between the instant centre of the knee and the patellar ligament. The average moment arm L_{H} of hamstring is expressed as a function of the knee flexion angle θ_{KF}, L_{H}=f (θ_{KF}), preferably defined by a polynomial fit function. That is:

L_{H}=[−3.008116131−0.04707811706*θ_{KF}+0.0003098140972*pow(θ_{KF},2)+1.867118879E−007*pow(θ_{KF},3)]/100

The force F_{PL} of the patellar ligament is calculated from dividing the quadriceps force F_{Q} by the mechanical advantage MA_{P} of the patella. That is:

F_{PL}=F_{Q}*MA_{P}

As MA_{P} at θ_{KF}=90° is approximately 0.6, F_{PL} is 1.67 times higher than F_{Q}.

The moments generated by the patellar ligament and by the hamstrings are calculated from the product of muscle forces and their moment arms, i.e. the moment about the knee instant centre produced by the quadriceps (via the patellar ligament)=M_{PL}.

M_{PL}=positive

M_{PL}=F_{PL}L_{PL}

Moment about the knee instant centre produced by the hamstrings=M_{H}

M_{H}=negative (from negative L_{H})

M_{H}=F_{H}L_{H}

The overall knee moment M_{K} is calculated from the sum of the muscle moments. That is:

M_{K}=M_{PL}+M_{H} (extending if positive, flexing if negative)

The external force, applied by the ground to the limb, is calculated by dividing the overall knee moment by the moment arm of the external force.

F_{Ex}=horizontal external force

F_{Ex}=M_{K}/[BH(0.285+0.039)]
Where: BH=body height (m); relative shank height≈0.285 BH, relative foot height≈0.039 BH; influence of plantar/dorsiflexion ignored.

The horizontal components of the patellar ligament and hamstrings forces, parallel to the tibial plateau, F_{PLx} and F_{Hx}, are calculated from their angles, θ_{PL} and θ_{H}.

F_{PLx}=F_{PL} sin θ_{PL} (positive via θ_{PL})

F_{Hx}=F_{H} sin θ_{H} (negative via θ_{H})

The horizontal net force of the shank is calculated from the sum of the horizontal force components of patellar ligament, hamstrings, and external force, considering that e.g. forces in anterior direction are positive and in posterior direction negative. Forces in anterior direction are balanced by the ACL, and forces in posterior direction are balanced by the PCL.

Fₓ₋ₙₑₜ=F_{PLx}+F_{Hx}+F_{Ex} (positive if forward to be compensated by ACL; negative if backward to be compensated by PCL)

The cruciate ligament forces, F_{ACL} and F_{PCL}, are calculated from the cruciate ligament angles, θ_{ACL} and θ_{PCL}.

F_{ACL}=force of ACL (output as positive value)

F_{ACL}=Fₓ₋ₙₑₜ/cos θ_{ACL}

F_{ACL}=force of PCL (output as positive value)

F_{PCL}=(−1)Fₓ₋ₙₑₜ/cos θ_{PCL}

As cruciate ligaments cannot be under tension at the same time, equations for decision making are required:

If Fₓ₋ₙₑₜ>0 (positive) then F_{ACL}>0 and F_{PCL}=0

If Fₓ₋ₙₑₜ<0 (negative) then F_{PCL}>0 and F_{ACL}=0

Thus:

F_{ACL}=H(Fₓ₋ₙₑₜ)(Fₓ₋ₙₑₜ/cos θ_{ACL})

F_{PCL}=[H(Fₓ₋ₙₑₜ)−1](Fₓ₋ₙₑₜ/cos θ_{PCL})
where H denotes the Heaviside function (unit step function); H(x)=(sgn(x)+1)/2, where sgn denotes the sign function.

The ACL loading data (F_{ACL}) is converted to an auditory, visual or tactile output signal to facilitate ACL overloading avoidance training with biofeedback to the subject. An auditory signal may be volume-coded or/and pitch-coded (the higher F_{ACL}, the louder or higher the tone). A visual signal may be brightness (gray-scale) or/and colour-coded (rainbow colours). Alternatively, the signal can be tactile, that is by way of a device producing vibrations.

A threshold can be included such that the subject wearing the sensors is alerted only of dangerous load above a pre-set threshold. Additional sensors recording knee rotation can enhance the biofeedback training, as the ACL is subjected to further tension on internal rotation of the shank. The biofeedback training applies to the PCL as well.

Muscle activity is plotted as:
F_{Q} and F_{H} against time and/or against θ_{KF}; and or dθ_{KF}/dt (time derivative of θ_{KF}=angular velocity ω_{KF} of shank)
ω_{KF}=(−1) dθ_{KF}/dt (multiplied by −1 such that extension is positive and flexion is negative)

Cumulative muscle activity is plotted as sum of activity data per muscle group over time. Comparison of synergistic muscle groups of right and left thigh for example (or any other body segment) for assessment of balance and unilateral overload.
Muscle power P against time (positive power=concentric contraction, negative power=eccentric contraction)

P_{Q}=M_{PL}ω_{KF}

P_{H}=M_{H}ω_{KF}

Muscle power is calculated from the product of muscle moment and time derivative of the knee flexion angle. Concentric contraction against eccentric contraction; the contraction ratio of a muscle indicates whether a muscle is subjected more to eccentric or concentric contraction.

Power across knee P_{K} against time

P_{K}=P_{Q}+P_{H}

The overall muscle energy is calculated from integrating the power across the knee with time.

Overall muscle energy E

E=∫P dt

Co-contraction refers to activating antagonistic muscle groups at the same time. Co-contraction increases the risk of joint injury due to joint overload as well as of muscle injuries if one of the muscle groups is further activated via the gamma-loop (i.e. via an overloaded ligament). If the ACL is overloaded (due to increased positive [forward-directed] Fₓ₋ₙₑₜ), then the hamstrings are activated via the gamma-loop and relieve the ACL of overload.

The magnitude of co-contraction CC is assessed by calculating the differential of the muscle forces:

CC=F_{Q}−F_{H} (positive if F_{Q} dominates over F_{H}; negative if F_{H} dominates over F₀)

Positive and negative CC are summed up individually over time and displayed e.g. as a bar chart.

The amount of co-contraction is calculated from the differential of muscle forces.

Co-contraction data (CC) are converted to an auditory, visual or tactile output signal, to facilitate co-contraction avoidance training be providing biofeedback. For example, for an auditory signal, the dominating muscle groups can be pitch coded:
higher pitch if F_{Q} dominates over F_{H}: indicates that the tension of the hamstrings should be reduced;
lower pitch if F_{H} dominates over F_{Q}: indicates that the tension of the quadriceps should be reduced;
whereas the magnitude of CC is volume-coded.

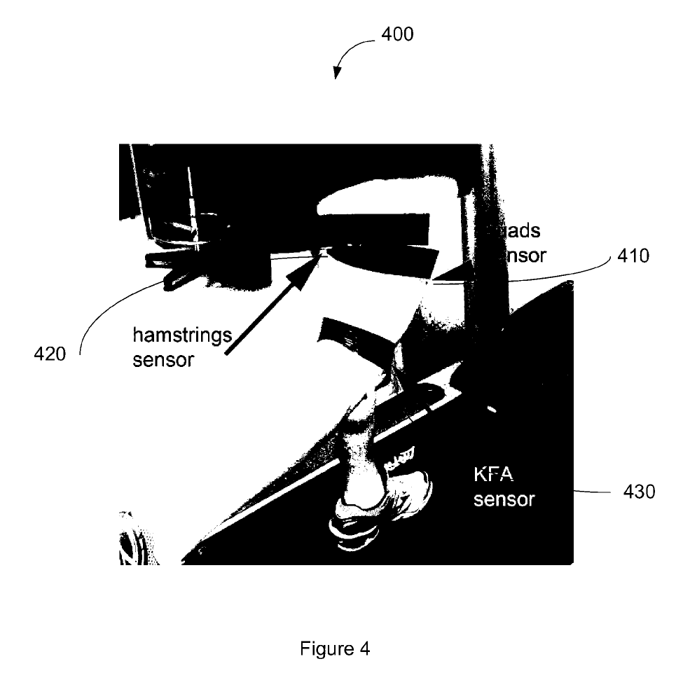

Referring now to FIG. 4, there is shown a functional prototype of a training aid 400 for monitoring and managing muscle activity and soft tissue loading. In accordance with the foregoing example the sensors are positioned to monitor muscle activity of the quadriceps and hamstrings and the flexion angle of the subject's knee.

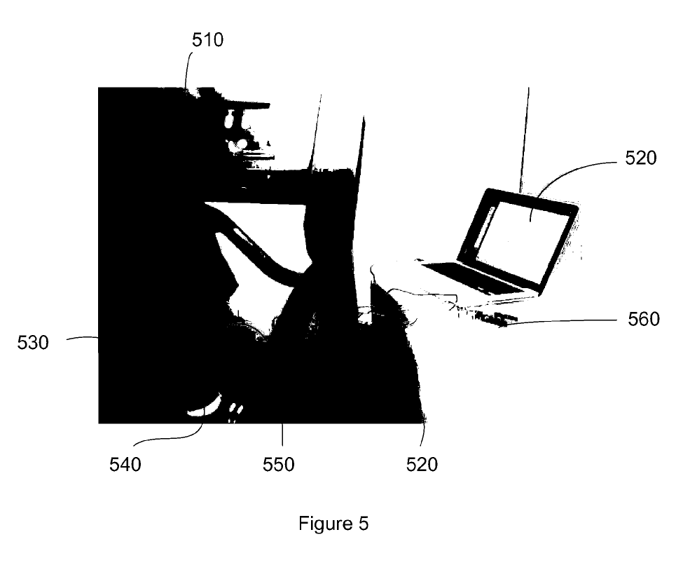

In FIG. 5, the subject 510 is shown running on treadmill 520 with the sensors 530, 540, 550 positioned on a body segment as described with reference to FIG. 4. The sensors 530, 540, 550 are connected to a circuit board and microcontroller 560 and the data is displayed on a laptop display 570.

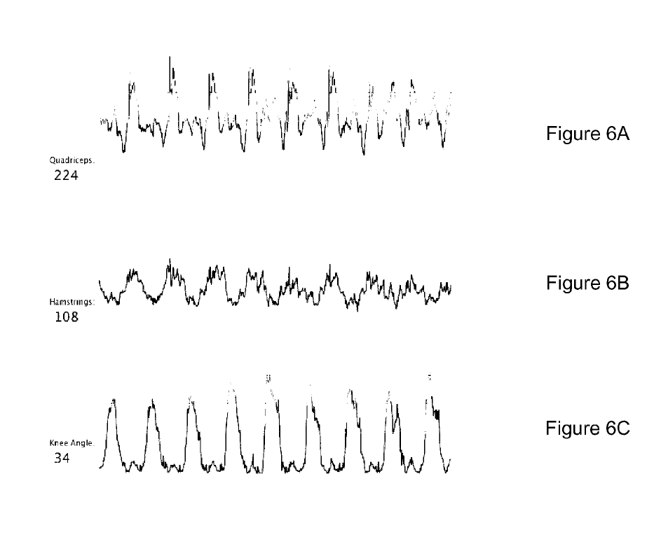

Referring now to FIGS. 6A to 6C, there is shown exemplary voltage signals detected by the sensors positioned at a body segment as described with reference to FIG. 4. FIG. 6A shows output from the sensor 410 positioned at the quadriceps, FIG. 6B shows output from the sensor 420 positioned at the hamstrings and FIG. 6C shows output from the sensor 430 positioned on the subject's knee for the purpose of monitoring the knee angle.

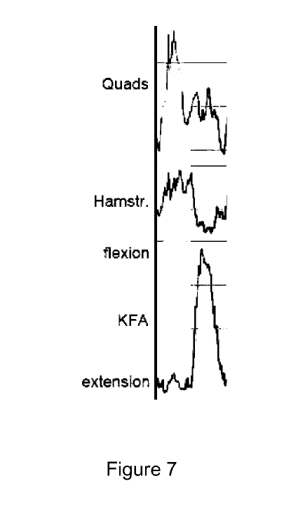

Referring now to FIG. 7, there is shown an extract of the data of FIGS. 6A to 6C. The white background shows that with the knee extended, in the stance phase of the running motion, the quadriceps and hamstrings co-contract. The grey background shows the swing phase of the running motion, i.e. with the knee flexed.

The sensors for measuring electric signals indicative of muscle activity and soft tissue loading levels can be strapped on to the body segment or applied using a suitable adhesive, or alternately incorporated into a garment to be donned by the subject. The garment may be athletic performance apparel such as a compression garment.

Such proposed intelligent compression garments are likely to stimulate and encourage physical activity by adding another aspect of interest to a program of physical exercise. Accordingly, the training aid proposed by the present invention is suitable to combat increasingly sedentary lifestyles which are commonly implicated in rising levels of obesity and the development of disorders such as cardiovascular disease, metabolic syndrome and type-II diabetes.

The integration of sensors in a compression garment means that an article of clothing becomes an indispensable training aid, providing real-time recognition of muscular activity. This information is processed and communicated back to the subject, effectively in real-time as auditory and/or visual signals to minimise injuries, reduce recovery time, and maximise training potential.

The method for monitoring and managing muscle activity and soft tissue loading of the present invention may be implemented using hardware, software or a combination thereof and may be implemented in one or more computer systems or processing systems capable of carrying out the above described functionality.

Although in the above described embodiments the invention is implemented primarily using computer software, in other embodiments the invention may be implemented primarily in hardware using, for example, hardware components such as an application specific integrated circuit (ASICs). Implementation of a hardware state machine so as to perform the functions described herein will be apparent to persons skilled in the relevant art. In other embodiments, the invention may be implemented using a combination of both hardware and software.

While the invention has been described in conjunction with a limited number of embodiments, it will be appreciated by those skilled in the art that many alternative, modifications and variations in light of the foregoing description are possible. Accordingly, the present invention is intended to embrace all such alternative, modifications and variations as may fall within the spirit and scope of the invention as disclosed.

1. A method for monitoring and managing muscle activity and soft tissue loading, the method including the following steps:
a) providing to a subject a plurality of sensors for measuring muscle activity and soft tissue loading levels;
b) directing the subject to undertake a program of exercise;
c) measuring muscle activity and soft tissue loading during the program of exercise;
d) comparing the measured muscle activity and soft tissue loading levels against calibrated muscle activity and soft tissue loading levels for the subject;
e) alerting the subject if the comparison of measured muscle activity and soft tissue loading levels against calibrated muscle activity and soft tissue loading levels indicates that a desirable level of muscle activity and/or soft tissue loading is being exceeded.

2. A method for monitoring and managing muscle activity and soft tissue loading according to claim 1, wherein the step of providing to a subject a plurality of sensors for measuring muscle activity and soft tissue loading levels includes providing at least two sensors configured to measure muscle activity and at least one sensor configured to measure a joint angle of a joint proximal to a muscle whose activity is to be measured by the at least two sensors.

3. A method for monitoring and managing muscle activity and soft tissue loading according to claim 2, wherein the soft tissue loading levels are determined as a function of the flexion angle of the proximal joint.

4. A method for monitoring and managing muscle activity and soft tissue loading according to claim 2 or 3, wherein the angle (θ_{ACL}) of an anterior cruciate ligament (ACL) of a lower limb to the tibial plateau, is expressed as a function of the knee flexion angle θ_{KF}, θ_{ACL}=f (θ_{KF}) and anterior cruciate ligament forces (F_{ACL}) are determined from an angle of the anterior cruciate ligament such that F_{ACL}=Fₓ₋ₙₑₜ/cos θ_{ACL}, wherein F x₋ₙₑₜ is a horizontal net force determined as a sum of horizontal force components of a patellar ligament, hamstrings, and external force, applied by a ground surface to the lower limb.

5. A method for monitoring and managing muscle activity and soft tissue loading according to claim 2 or 3, wherein the angle (θ_{ACL}) of a posterior cruciate ligament (PCL) of a lower limb to the tibial plateau, is expressed as a function of the knee flexion angle θ_{KF}, θ_{PCL}=f (θ_{KF}) and posterior cruciate ligament forces (F_{PCL}) are determined from an angle of the posterior cruciate ligament such that F_{PCL}=(−1)Fₓ₋ₙₑₜ/cos θ_{PCL}, wherein F x₋ₙₑₜ is a horizontal net force determined as a sum of horizontal force components of a patellar ligament, hamstrings, and external force, applied by a ground surface to the lower limb.

6. A method for monitoring and managing muscle activity and soft tissue loading according to any one of claim 2 or 3, where a simultaneous contraction of agonist and antagonist muscles is determined from a differential of muscle forces such that CC=F_{Q}−F_{H} wherein, F_{Q}=quadriceps force and F_{H}=hamstrings force as determined from sensed voltage signals.

7. A method for monitoring and managing muscle activity and soft tissue loading according to any one of claims 1 to 6, wherein the step of comparing the measured muscle activity and soft tissue loading levels against calibrated muscle activity and soft tissue loading levels occurs in real-time.

8. A method for monitoring and managing muscle activity and soft tissue loading according to any one of claim 1 or 7, wherein the step of alerting the subject if the comparison of measured muscle activity and soft tissue loading levels against calibrated muscle activity and soft tissue loading levels indicates that a desirable level of muscle activity and/or soft tissue loading is being exceeded occurs in real-time.

9. A method for monitoring and managing muscle activity and soft tissue loading according to any one of claims 1 to 8, wherein a determination of the calibrated muscle activity and soft tissue ligament loading levels includes directing the subject to perform a series of movements and measuring the muscle activity and soft tissue loading levels of the subject for each movement to build a baseline profile for the subject against which muscle activity and soft tissue loading levels measured during a program of exercise will be compared.

10. A method for monitoring and managing muscle activity and soft tissue loading according to claim 9, wherein the step of calibrating the muscle activity and soft tissue loading levels for the subject involves measuring a maximum voluntary contraction of a quadricep and a hamstring respectively corresponding to at least three different knee flexion angles.

11. A method for monitoring and managing muscle activity and soft tissue loading according to any one of claims 1 to 10, wherein the step of alerting the subject if the comparison of measured muscle activity and soft tissue loading levels against calibrated muscle activity and soft tissue loading levels indicates that a desirable level of muscle activity and/or soft tissue loading levels is being exceeded includes providing an auditory, visual or tactile alert to the subject.

12. A method for monitoring and managing muscle activity and soft tissue loading according to any one of claims 1 to 11, wherein the step of providing to a subject a plurality of sensors for measuring muscle activity and soft tissue loading levels involves providing a garment incorporating the sensors to the subject.

13. A system for monitoring and managing muscle activity and soft tissue loading, the system including:
a) a plurality of sensors for measuring electric signals indicative of muscle activity and soft tissue loading levels;
b) a processor configured to receive the electric signals and covert them to muscle activity and soft tissue loading values, the processor further configured to compare the muscle activity and soft tissue loading values against calibrated muscle activity and soft tissue loading levels for a subject; and
c) an alert module to alert the subject if the comparison of measured muscle activity and/or soft tissue loading levels against calibrated muscle activity and soft tissue loading levels indicates that a desirable level of muscle activity and/or soft tissue loading is being exceeded.

14. A system for monitoring and managing muscle activity and soft tissue loading according to claim 13, wherein the plurality of sensors for measuring electric signals indicative of muscle activity and soft tissue loading levels include at least three sensors.

15. A system for monitoring and managing muscle activity and soft tissue loading according to claim 14, wherein the at least three sensors are positioned on the subject at the following locations:
a) a first position which will contact an anterior, medial or posterior skin surface of a body segment of the subject;
b) a second position which will contact a remaining contact an anterior, medial or posterior skin surface of the body segment; and
c) a third position which will contact an anterior, posterior, medial or lateral skin surface of a joint proximal to the body segment.

16. A system for monitoring and managing muscle activity and soft tissue loading according to claim 15, wherein the sensors in the first and second positions are configured to measure muscle activity of the body segment.

17. A system for monitoring and managing soft tissue loading according to claim 15 or 16, wherein the sensor in the third position is configured to measure the angle of the joint proximal to the body segment.

18. A system for monitoring and managing muscle activity and soft tissue loading according to any one of claims 13 to 17, wherein the plurality of sensors include a combination of pressure sensors and electrogoniometric sensors.

19. A system for monitoring and managing muscle activity and soft tissue loading according to claims 13 to 18, wherein the plurality of sensors for measuring electric signals indicative of muscle activity and soft tissue loading levels are incorporated into a garment to be donned by the subject.

20. A system for monitoring and managing muscle activity and soft tissue loading according to claim 19, wherein the garment is a compression garment.

21. A training aid for monitoring and managing muscle activity and soft tissue loading, the training aid including:
a) a garment incorporating a plurality of sensors for measuring electric signals indicative of muscle activity and soft tissue loading levels;
b) processor configured to receive the electric signals and covert them to muscle activity and soft tissue loading values, the processor further configured to compare the muscle activity and soft tissue loading values against calibrated muscle activity and soft tissue loading levels for a subject; and
c) an alert module to alert the subject if the comparison of measured muscle activity and/or soft tissue loading levels against calibrated muscle activity and soft tissue loading levels indicates that a desirable level of muscle activity and/or soft tissue loading is being exceeded.

22. A training aid for monitoring and managing muscle activity and soft tissue loading according to claim 21, wherein the processor and alert module are provided in a portable telecommunications device.

23. A training aid for monitoring and managing muscle activity and soft tissue loading according to claim 21 or 22, wherein the garment incorporating a plurality of sensors is a compression garment.

24. A method for monitoring and managing muscle activity and soft tissue loading substantially as hereinbefore described with reference to the example.
